(19) United States
(12) Patent Application Publication
(10) Pub. No.: US 2005/0031712 A1
(43) Pub. Date: Feb. 10, 2005

(54) Agent for suppressing or preventing gastralgia, and composition for food or beverage

(76) Inventor: Kaneko Nagai, Osaka (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(21) Appl. No.: 10/912,049

(22) Filed: Aug. 6, 2004

(30) Foreign Application Priority Data
Aug. 8, 2003 (JP) 2003-289853

(51) Int. Cl. A61K 35/78

(52) U.S. Cl. 424/729; 424/761; 424/765; 424/771; 424/774; 424/750

(57) ABSTRACT

It is an object of the present invention to provide to formulations for suppressing or preventing gastralgia that occurs due to the consumption of compositions for food or beverage containing green tea, and to compositions for food or beverage containing said formulations. An agent for suppressing or preventing gastralgia contains mulberry. Also, a composition for food or beverage contains field horsetail, mulberry, mugwort, and green tea, that composition for food or beverage further contains bamboo grass, that composition for food or beverage further contains ground ivy, that composition for food or beverage further contains persimmon leaves, and that composition for food or beverage is in the form of powder or granules.

### FIELD OF THE INVENTION

The present invention relates to formulations for suppressing or preventing gastralgia that occurs due to the consumption of compositions for food or beverage containing green tea, and to compositions for food or beverage containing said formulations.

### BACKGROUND OF THE INVENTION

Conventionally, many health food products that employ medicinal herbs have been developed and used.

In particular, field horsetail is a hardy plant, which can be inferred from the fact that it is known as a weed, that has recently garnered attention because it includes an abundant amount of trace elements that humans tend to have insufficiencies in, and lowers blood pressure due to the action of silicon, for example.

Many proposals have been made for food items that blend field horsetail with other medicinal herbs, such as blending green tea with field horsetail (see Japanese Patent No. 2933774 (published May 28, 1999)).

However, this has the problem that gastralgia occurs easily when taken, possibly due to the presence of the green tea component. The problem of gastralgia is a fatal drawback for drugs or health food products that must be taken repeatedly. Firstly, this is because repeated dosing is itself difficult, but also, the effect of such drugs or health food products is halved when one must force themselves to take such drugs or health food products while feeling an aversion toward them.

### SUMMARY OF THE INVENTION

The inventor of the present application arrived at the present invention after conducting extensive research aimed at solving the foregoing problem. It is an object of the present invention to provide formulations for suppressing or preventing gastralgia that occurs due to the consumption of compositions for food or beverage containing green tea, and compositions for food or beverage containing said formulations.

The foregoing object is achieved through an agent for suppressing or preventing gastralgia that contains mulberry, a composition for food or beverage that contains field horsetail, mulberry, mugwort, and green tea, that composition for food or beverage further containing bamboo grass, that composition for food or beverage further containing ground ivy, that composition for food or beverage further containing persimmon leaves, and these compositions for food or beverage in the form of powder or granules.

With the agent for suppressing or preventing gastralgia according to the present invention, it is possible to effectively suppress or prevent gastralgia that occurs due to the consumption of compositions for food or beverage that contain green tea.

The composition for food or beverage according to the present invention has an excellent blood pressure lowering action, and also has the benefit that it is fast acting. Also, because gastralgia does not occur, even patients with easily irritated stomachs or intestines can take the composition repeatedly with ease without feeling aversion toward eating or drinking. Moreover, it can be easily eaten and drunken and repeatedly dosed.

### BRIEF DESCRIPTION OF THE DRAWINGS

No Drawings

### DESCRIPTION OF THE PREFERRED EMBODIMENTS

Mulberry (Morus L.) used in the present invention is a dicotyledon belonging to the Moraceae, and generally includes deoxynojirimycin, calcium, β-carotene (vitamin A), γ-amino acid, flavonoids, potassium, iron, and amino acid, for example. There are many types of mulberry, and without any particular regard to type, representative examples thereof include: white mulberry (Morus alba L.; Morus atropurpure Roxb.), Korean mulberry (Morus australis Poir.; Morus acidosa Griff.), Japanese mulberry (Morus bombycis Koidz.; Morus alba L. var. stylosa Bur.; Morus japonica Bailey nonSieb.), hachijo kuwa (Morus lagayamae Koidz.), large-leaved mulberry (Morus latifolia (Bur.) Poir.; Morus alba L. var. latifolia Bur.; Morus multicaulis Perr.; Morus alba L. var. multicaulis Loud.), Mongolian mulberry (Morus mongolica (Bur.) Schneid.; Morus alba var. ongolica Bur.), black mulberry (Morus nigra L.), and red mulberry (Morus rubra L.). Of these mulberries, white mulberry (Morus alba L.; Morus atropurpure Roxb.) and Japanese mulberry (Morus bombycis Koidz.; Morus alba L. var. stylosa Bur.; Morus japonica Bailey nonSieb.), and strains of these for cultivation, are preferable from the standpoint of raw material supply. It is possible to use the whole mulberry plant except for the root, the leaves and the stem, for example, but it is preferable that the whole plant is used. Also, mulberry that has been cultivated or gathered using little or no pesticides is preferable because such plants contain few harmful chemically synthesized substances and the water-soluble or lipid-soluble vitamins, minerals, and cellulose created by the plants themselves, and the energy created by these, can be effectively obtained.

Using mulberry, gastralgia that most likely occurs due to the caffeine in food and beverages containing green tea can be effectively suppressed or prevented. In addition, mulberry also has the effect of preventing diabetes, lowers blood pressure and cholesterol, suppresses an increase in fat, relieves atopic dermatitis, increases helpful intestinal bacteria, prevents cancer, and prevents arteriosclerosis.

In the present invention, there is no particular limitation to the mulberry content when mulberry is used as an agent for suppressing or preventing gastralgia, and it is preferably from 5 to 100 wt %, and more preferably from 10 to 95 wt %. When mulberry is contained in compositions for food or beverage, the mulberry content is preferably from 7 to 50 wt %, more preferably from 9 to 30 wt %, and particularly preferably from 10 to 25 wt %.

Field horsetail (Equisetem arvense L.) used in the present invention is a perennial of the Equisetaceae, and generally includes dietary fiber, equisetonin, silicon, calcium, potassium, magnesium, zinc, iron, minerals, vitamins A, B, C, D, E, and K, folic acid, nicotinic acid, and pantothenic acid, for example. Although it is possible to use the whole plant, the whole plant except for the root, the leaves, and the stem, for example, it is preferable that the whole plant is used. Field horsetail that has been cultivated or gathered using little or no pesticides is preferable because it has a pleasant scent and is eaten and drunken with ease.

Using field horsetail, a hypotensive action is attained because the silicon contained in the field horsetail traps hydrogen ions in blood. The hypotensive action of field horsetail runs little risk of rapidly lowering only the systolic blood pressure and thereby narrowing the range between the systolic blood pressure and the diastolic blood pressure like that of commonly used hypotensive agents. In other words, it also has the effect of lowering the diastolic blood pressure at the same time as the systolic blood pressure and stabilizes blood pressure, and thus has the advantage that it places little burden on the body. Field horsetail is also effective in preventing cancer, inhibiting anal polyps, and preventing diabetes, and it can also be expected to increase immunity, eliminate constipation, improve dermatitis, improve atopic dermatitis, function as a diuretic, strengthen blood vessels of the heart, reduce fever, relieve pain after fractures, reduce coughing, prevent phlegm, suppress asthma, prevent and improve liver disease, reduce edema, and dissolve kidney and bladder stones.

The content of field horsetail in the composition for food or beverage of the present invention is preferably 1 to 50 wt %, more preferably 2 to 40 wt %, and particularly preferably 3 to 30 wt %.

Mugwort used in the present invention is a perennial of the Asteraceae, and generally includes tannin, p-carotene (vitamin A), calcium, iron, potassium, dietary fiber, folic acid, vitamins B2, C and D, sodium, flavonoids, and essential oil constituents such as thujone and cineole. Although it is possible to use the whole plant, the whole plant other than the root, the leaves, and the stem, for example, it is preferable that the whole plant is used. Mugwort that has been cultivated or gathered using little or no pesticides is preferable because such plants contain few harmful chemically synthesized substances and the water-soluble or lipid-soluble vitamins, minerals, and cellulose created by the plants themselves, and the energy created by these, can be effectively obtained. Also, mugwort produced in Japan is preferable because Japanese will find its scent familiar when eating it.

When used, mugwort can be expected to prevent heartburn, eliminate constipation, lower blood pressure, improve dyspepsia, stop bleeding, function as a diuretic, prevent and improve allergy-related ailments, have anti-inflammatory action, sterilize, treat eyestrain, improve poor circulation, prevent and relieve gynecological ailments such as menstrual pain, prevent and improve neuralgia, prevent and improve rheumatism, relieve pain associated with hemorrhoids, relieve back pain, improve metabolic disorders, purify blood, increase the blood amount, have a triglyceride and cholesterol lowering action, prevent arteriosclerosis, and prevent cancer.

The content of mugwort in the composition for food or beverage of the present invention is preferably 5 to 50 wt %, more preferably 7 to 40 wt %, and particularly preferably 8 to 20 wt %.

The green tea used in the present invention is a plant of the Theaceae, and generally includes water-soluble components such as catechin, caffeine, amino acids, vitamins B1, B2, C, P, and V, and saponin, as well as lipid-soluble components such as β-carotene (vitamin A), dietary fiber, vitamin E, chlorophyll, protein, and fluorine, and minerals such as potassium, calcium, phosphorous, magnesium, and iron. Green tea that has been cultivated or gathered using little or no pesticides is preferable because such plants contain few harmful chemically synthesized substances and the water-soluble or lipid-soluble vitamins, minerals, and cellulose created by the plants themselves, and the energy created by these, can be effectively obtained.

When used, green tea can be expected to have an anticancer effect, improve allergy-related ailments, lower blood cholesterol and triglycerides, have a skin-whitening action and an anti-ageing action, prevent arteriosclerosis, prevent diabetes, lower blood pressure, prevent the common cold, prevent food poisoning, improve the balance of intestinal bacteria, eliminate stress, prevent hangovers, strengthen teeth, inhibit fatigue, accelerate fat burning, relieve drowsiness, maintain cells, and accelerated recovery from wounds.

The content of green tea in the composition for food or beverage of the present invention is preferably 10 to 60 wt %, more preferably 20 to 50 wt %, and particularly preferably 30 to 40 wt %.

The bamboo grass (sasa) used in the present invention is a plant of the Gramineae, and generally include chlorophyll, amino acid, sasa polysaccharide, protein, vitamins, and flavones. One example of bamboo grass is kuma-zasa (Sasa veitchii). Examlpes of kuma-zasa include kuma-zasa in a narrow sense, kumaki-zasa, chimaki-zasa, nikko-zasa, ooba-zasa, and miyako-zasa. Large-sized plants are preferable because their leaf portions are large and can be processed efficiently, and kumaki-zasa and chimaki-zasa are preferable because they have a long history of being used for food. Although it is possible to use the whole plant, the whole plant other than the root, the leaves, and the stem, for example, it is preferable that the leaf portions is used. Bamboo grass that has been cultivated or gathered using little or no pesticides is preferable because such plants contain few harmful chemically synthesized substances and the water-soluble or lipid-soluble vitamins, minerals, and cellulose, for example, created by the plants themselves, and the energy created by these, can be effectively obtained.

When used, bamboo grass can be expected to purify blood, suppress cancer, prevent gastric ulcers and duodenal ulcers (protect the mucous membrane), have an anti-allergy effect, regenerate cell membranes, lower blood pressure, lower cholesterol, prevent liver disease, repair beta cells, improve atopic dermatitis, prevent and improve allergy-related ailments, prevention and improve pyorrhea alveolaris, have an antimicrobial effect and an anti-inflammatory effect, and increase immunity.

The content of bamboo grass in the composition for food or beverage of the present invention is preferably 5 to 50 wt %, more preferably 6 to 40 wt %, and particularly preferably 7 to 30 wt %.

Ground ivy (Glechoma hederacea) employed in the present invention is a perennial belonging to the Labiatae, and is also commonly known as Creeping Charlie. Ground ivy generally includes sesquiterpenes, flavonoids, peppermint-like essential oil compounds, glechomine bitter principle, and saponins, for example. Although it is possible to use the whole plant, the whole plant except for the root, the leaves, the stem, and the root, for example, it is preferable that the whole plant is used. Ground ivy that has been cultivated or gathered using little or no pesticides is preferable because such plants contain few harmful chemically synthesized substances and the water-soluble or lipid-soluble vitamins, minerals, and cellulose, for example, created by the plants themselves, and the energy created by these, can be effectively obtained.

When used, ground ivy can be expected to function as a diuretic, prevent kidney disorders, prevent diabetes, have a anti-inflammatory effect, prevent jaundice, prevent the formation of stones, treat chronic colds, lower blood sugar, dissolve lipids, lower cholesterol and triglycerides, lower diastolic blood pressure, reduce fever, improve bronchitis, improve lung disease, treat nervousness in children, have benefit to persons with a weak constitution, increase vigor, and have a relaxing effect.

The content of ground ivy in the composition for food or beverage of the present invention is preferably 5 to 50 wt %, more preferably 6 to 40 wt %, and particularly preferably 7 to 30 wt %.

The persimmon leaves employed in the present invention are the leaves of plants belonging to the Ebenaceae. Persimmon leaves have a particularly large vitamin C (ascorbic acid) content when compared to other plants, and also generally includes tannin, catalase, kaempferol-3-glucoside, and quercetin-3-glucoside, for example. Persimmon leaves of trees that have been cultivated or gathered using little or no pesticides are preferable because such leaves contain few harmful chemically synthesized substances and the water-soluble or lipid-soluble vitamins, minerals, and cellulose, for example, created by the trees themselves, and the energy created by these, can be effectively obtained.

When used, persimmon leaves can be expected to have a skin-whitening effect, prevent scurvy, lower blood pressure, prevent arteriosclerosis, prevent heart disease, prevent the common cold, prevent hangovers, stop internal bleeding, and prevent diseases of the kidneys.

The content of persimmon leaves in the composition for food or beverage of the present invention is preferably 5 to 50 wt %, more preferably 6 to 40 wt %, and particularly preferably 7 to 30 wt %.

The agent for suppressing or preventing gastralgia of the present invention, which is characterized in that it contains mulberry, can simultaneously contain other pharmaceutical agent components or components such as exipients in addition to the mulberry, as long as these do not interfere with the effects of the mulberry, and natural materials are preferable. There are no particular limitations regarding the form of the suppressing or preventing agent, and it may take a solid form such as powder, granules, tablets, or capsules, or it may take a liquid form, but from the standpoint of the ease with which the agent can be taken, preservability, and portability, a solid form such as powder, granules, tablets, or capsules is preferable, and powder, granules, and capsules are particularly preferable. Although the agent can be taken as is, it can also be used added into other food items.

The composition for food or beverage according to the present invention contains field horsetail, mulberry, mugwort, and green tea. This composition can simultaneously contain other pharmaceutical agent components or components such as exipients as long as these do not interfere with the effects of the raw material components, and in this case, natural material are preferable.

The preferable content ratios of the various components in the composition for food or beverage according to the present invention are as outlined above, and although there are no particular restrictions regarding content ratios between components, in order to effectively achieve the effect of suppressing or preventing gastralgia resulting from green tea it is preferable that the ratio of mulberry:green tea is from 3:4 to 1:4, and more preferably is from 2:3 to 1:3. Also, to appropriately adjust the constipation eliminating effect of field horsetail, it is preferable that ratio of field horsetail: mugwort is from 5:1 to 1:3, and more preferably is from 3:1 to 1:2.

In addition to the various effects of field horsetail, mulberry, mugwort, and green tea that are mentioned above, the composition for food or beverage also has the excellent benefits that the speed at which it begins to lower blood pressure is significantly faster than that of food products containing the three components of field horsetail, mugwort, and green tea, and moreover, gastralgia that is characteristic of food products containing green tea does not occur.

The composition for food or beverage according to the present invention can further contain bamboo grass in addition to the above.

This composition for food or beverage has the excellent benefit that it improves dyspepsia and prevents prurigo (itchiness) in addition to the effects mentioned above.

The composition for food or beverage according to the present invention can also contain ground ivy in addition to the above.

This composition for food or has the excellent benefit that it eliminates stress, prevents edginess, and dissolves stones in addition to the effects mentioned above.

The composition for food or beverage according to the present invention can also contain persimmon leaves in addition to the above.

This composition for food or has the excellent benefit that the blood pressure lowering action is increased, in addition to the effects mentioned above.

There are no particular limitations regarding the form of these compositions for food or beverage according to the present invention, and they may take a solid form such as powder, granules, tablets, or capsules, or they may take a liquid form, but from the standpoint of the ease with which they can be taken, preservability, and portability, a solid form such as powder, granules, tablets, or capsules is preferable, and powder, granules, and capsules are particularly preferable.

The average particle size of the powder is preferably 100 to 200 mesh. This is because particles larger than those that pass through 200 mesh scatter little when working with or administrating them, nutritional components can be utilized without destroying the cells of the raw material components, they are not likely to stick to one's throat when taken, and they quickly dissolve in water, and if the particles pass through 100 mesh, then they are not likely to catch on one's throat during administration.

These raw materials are cleaned and dried (e.g. hot-air dried or kiln-dried), for example, and then, where necessary, sterilized by microwave or steam and pulverized through a method such as air flow pulverization using a jet mill, pounding with a mallet, and grinding. Drying is carried out such that the water content is preferably not more than 8 wt %, and more preferably not more than 6 wt %.

If the composition for food or beverage according to the present invention is in the form of a powder, then the pulverized particles can be used as they are, but if it is in the form of granules, then it can be produced by adding a starch aqueous solution or the like to the pulverized particles to form granules. If in the form of granules, then it is preferable that the granules are adjusted to preferably pass through 10 mesh and more preferably to pass through 13 mesh, from the standpoint of foreign object removal and the ease of administration, for example. It should be noted that if provided as granules in this way, then it is not necessary to filter the particles before granulation to the size mentioned above.

The agent for suppressing or preventing gastralgia and the composition for food or beverage according to the present invention can be made available for drinking or eating in their present form, and can also be made available for eating or drinking by adding them to food or beverage products.

### WORKING EXAMPLE 1

### Example of a Blend of Four Components

| | |
|---|---|
| Field Horsetail powder (average 100 to 200 mesh) | 30 g |
| Mulberry powder (average 100 to 200 mesh) | 20 g |
| Mugwort powder (average 100 to 200 mesh) | 10 g |
| Green Tea powder (average 100 to 200 mesh) | 40 g |

The powders obtained in the manner discussed above were sent through a sieve to obtain a composition for food or beverage made of powder whose average mesh is 100 to 200 mesh.

This composition for food or beverage was administered to hypertensive patients whose systolic blood pressure was 200 mmHg and diastolic blood pressure was 110 mmHg. The day after administration the patients' systolic blood pressure had already dropped to 140 mmHg, and their diastolic blood pressure, which should be difficult to lower using normal hypotensive agents, had become 65 mmHg.

Furthermore, this composition for food or beverage did not result in gastralgia, and thus even patients with weak stomachs or intestines showed no aversion to eating or drinking the composition and repeated administration was easy.

### COMPARATIVE EXAMPLE 1

The same composition for food or beverage as that of Working Example 1 was produced using the same raw materials, with the exception of mulberry.

| | |
|---|---|
| Field Horsetail powder (average 100 to 200 mesh) | 5 g |
| Mugwort powder (average 100 to 200 mesh) | 45 g |
| Green Tea powder (average 100 to 200 mesh) | 50 g |

This composition for food or beverage was administered to hypertensive patients with a systolic blood pressure of 200 mmHg, and three days after administration the patients' systolic blood pressure had become 180 mmHg, and after one week this has dropped to between 150 to 160 mmHg.

This composition for food or beverage is prone to causing gastralgia, and patients whose stomachs or intestines in particular showed aversion to eating or drinking and repeated administration was difficult.

### WORKING EXAMPLE 2

The particles of an average mesh of 100 to 200 mesh that were obtained in Working Example 1 were kneaded within a granule forming machine while an aqueous solution of 3 wt % matsunorin (potato starch grain) was added thereto by blowing, at a ratio of 400 g to 1 kg of the powder. When gummy particles appeared, drying was performed using drying racks (drying chamber temperature 50°, 48 to 72 hours). The result was then sent through a sieve to select granules that pass through 14 mesh, thereby yielding granule-shaped articles of the present invention.

### WORKING EXAMPLE 3

### Example of a Blend of Five Components

A composition for food or beverage according to the present invention was produced by adding, in addition to the average 100 to 200 mesh powder obtained in Working Example 1, 20 g of kuma-zasa powder (average 100 to 200 mesh) that was obtained in the same manner.

This composition for food or beverage, in addition to having a blood pressure lowering action, demonstrated an excellent anti-allergy effect, a dyspepsia alleviating action, and a prurigo preventing action.

### WORKING EXAMPLE 4

### Example of a Blend of Six Components

A composition for food or beverage according to the present invention was produced by adding, in addition to the average 100 to 200 mesh powder obtained in Working Example 1, 10 g of kuma-zasa powder (average 100 to 200 mesh) and 10 g of ground ivy powder (average 100 to 200 mesh) that were obtained in the same manner.

This composition for food or beverage, in addition to having a blood pressure lowering action, demonstrated an excellent anti-allergy effect, a dyspepsia alleviating action, and a prurigo preventing action, and moreover, eliminates stress, prevents edginess, and has the effect of dissolving stones.

### WORKING EXAMPLE 5

### Example of a Blend of Seven Components

A composition for food or beverage according to the present invention was produced by adding, in addition to the average 100 to 200 mesh powder obtained in Working Example 1, 10 g of kuma-zasa powder (average 100 to 200 mesh), 10 g of ground ivy powder (average 100 to 200 mesh), and 10 g of persimmon leaf powder (average 100 to 200 mesh) that were obtained in the same manner.

This composition for food or beverage, in addition to having a blood pressure lowering action, demonstrated an excellent anti-allergy effect, a dyspepsia alleviating action, and a prurigo preventing action, and moreover, eliminates stress, prevents edginess, and has the effect of dissolving stones.

Compositions for food or beverage according to the present invention were produced as discussed above using the following composition ratios.

### WORKING EXAMPLE 6

### Example of a Blend of Five Components

field horsetail log, mulberry 20 g, mugwort 20 g, green tea 40 g, kuma-zasa 10 G

Like Working Example 3, this composition for food or beverage demonstrated blood pressure lowering action as well as an excellent anti-allergy effect, a dyspepsia alleviating action, and a prurigo preventing action.

### WORKING EXAMPLE 7

### Example of a Blend of Seven Components

field horsetail 10 g, mulberry 10 g, mugwort 10 g, green tea 30 g, kuma-zasa 20 g, ground ivy 10 g, persimmon leaves 10 g

Like Working Example 5, this composition for food or beverage demonstrated an excellent anti-allergy effect, a dyspepsia alleviating action, and a prurigo preventing action in addition to a particularly excellent blood pressure lowering action.

The invention may be embodied in other forms without departing from the spirit or essential characteristics thereof. The embodiments disclosed in this application are to be considered in all respects as illustrative and not limiting. The scope of the invention is indicated by the appended claims rather than by the foregoing description, and all changes which come within the meaning and range of equivalency of the claims are intended to be embraced therein.

1. An agent for suppressing or preventing gastralgia, comprising mulberry.

2. A composition for food or beverage, comprising field horsetail, mulberry, mugwort, and green tea.

3. The composition for food or beverage according to claim 2, further comprising bamboo grass.

4. The composition for food or beverage according to claim 2, further comprising ground ivy.

5. The composition for food or beverage according to claim 3, further comprising ground ivy.

6. The composition for food or beverage according to claim 2 further comprising persimmon leaves.

7. The composition for food or beverage according to claim 3, further comprising persimmon leaves.

8. The composition for food or beverage according to claim 4, further comprising persimmon leaves.

9. The composition for food or beverage according to claim 5, further comprising persimmon leaves.

10. The composition for food or beverage according to claim 2, wherein said composition is in the form of powder or granules.

11. The composition for food or beverage according to claim 3, wherein said composition is in the form of powder or granules.

12. The composition for food or beverage according to claim 4, wherein said composition is in the form of powder or granules.

13. The composition for food or beverage according to claim 5, wherein said composition is in the form of powder or granules.

14. The composition for food or beverage according to claim 6, wherein said composition is in the form of powder or granules.

15. The composition for food or beverage according to claim 7, wherein said composition is in the form of powder or granules.

16. The composition for food or beverage according to claim 8, wherein said composition is in the form of powder or granules.

17. The composition for food or beverage according to claim 9, wherein said composition is in the form of powder or granules.
